(19) United States
(12) Patent Application Publication
(10) Pub. No.: US 2003/0050582 A1
(43) Pub. Date: Mar. 13, 2003

(54) Head support

(76) Inventor: Darrell Maitland Poole, Auckland (NZ)

Correspondence Address: JACOBSON HOLMAN PLLC, 400 SEVENTH STREET N.W., SUITE 600, WASHINGTON, DC, 20004, US

(21) Appl. No.: 10/217,164

(22) Filed: Aug. 13, 2002

Related U.S. Application Data
(63) Continuation of application No. 09/737,557, filed on Dec. 18, 2000, now abandoned.
Application No. 09/737,557 is a 371 of international application No. PCT/NZ00/00106, filed on Jun. 21, 2000.

(30) Foreign Application Priority Data
Jun. 21, 1999 (NZ) 336358

(51) Int. Cl. A61F 5/00

(52) U.S. Cl. 602/17; 602/18

(57) ABSTRACT

The invention relates to head or neck supports which support the head or neck in a tilted back position and comprises or includes:

a) a harness locatable to the upper body portions of a person,

b) a rigid member extending from a region of said harness stabilised in respect of and by the use of the harness relative to the body,

wherein, when in use, said harness is attached to the body of a person, said rigid member extending therefrom to provide a support region thereof which when said person is in a substantially upright position, and the head is tilted backwards to allow viewing of an object above the horizontal, said support region comes in contact with at least part of the back of the head and provides at least vertical support to the head.

### FIELD OF INVENTION

The present invention relates to improvements in or relating to head supports. More particularly but not exclusively it relates to head or neck supports which support the head or neck in a tilted back position, and/or to provide support for viewing objects above the horizontal.

### BACKGROUND TO THE INVENTION

In activities such as climbing, fruit picking, plastering, painting or attending air shows or the like where a person is, for long durations, required or desired to have his/her head tilted back so as to allow them to look upwardly or at least above the horizontal, such tilting of the head can only be sustained for a limited period of time. Although muscle strength to support a head in a tilted position does vary from person to person, after a while anyone will start to feel the strain in having to maintain the head in such a condition. In activities such as climbing where a belayer is positioned below the climber and whose function is to maintain the tension in the rope which is attached to the climber, it is essential that this person keeps a close eye on the progress of the climber as he/she ascends or descends. Failure to maintain a substantially constant eye on the progress of the climber could result in a sufficient amount of slack in the rope which if the climber falls, could result in a fall, which may cause injury or death.

It is an object of the present invention to provide a head support which will at least go some way towards providing a means of supporting the head or neck of a person in a tilt back condition or which will at least provide the public with a useful choice.

As used herein, “the horizontal” describes that field of view of the eyes in the plane substantially perpendicular to the body of the user, which is considered as being in the “upright” position. The “upright” position may include a position which is not strictly vertical or upright as in conventionally understood. For example, when in a standing, leaning back, or sitting position, the body is defined as being in the “upright” position. The “horizontal” view is that with the eyes facing substantially straight ahead. In viewing an object “above the horizontal” the head and/or neck will require tilting backwards somewhat from this position.

“Harness” as used herein may also include a backpack or other system capable of being worn by or attached to a user, and supporting or appending to or functioning as a head or neck support as described.

### BRIEF DESCRIPTION OF THE INVENTION

Accordingly in a first aspect the present invention consists in a head support comprising or including:

a) a harness locatable to the upper body portions of a person

b) a rigid member extending from a region of said harness stabilised in respect of and by the use of the harness relative to the body

wherein, when in use, said harness is attached to the body of a person, said rigid member extending therefrom to provide a support region thereof which when said person is in a substantially upright position, and the head is tilted backwards to allow viewing of an object above the horizontal, said support region comes in contact with at least part of the back of the head and provides at least vertical support to the head.

Preferably said rigid member has a vertically extending portion which, in use extends substantially vertically and parallel with the general upright position of said user, and an outwardly extending portion which extends, in use from said vertically extending portion, outwardly from the body of a user wherein at or near the distal end of said outwardly extending portion said head supporting region is provided.

Preferably said rigid member is secured to said harness at said vertically extending portion thereof.

Preferably said rigid member is provided with padding in those regions which, when said head support is in use, is in contact with the body of the user.

Preferably said harness is secured to the vertically extending portion of said rigid member to, when said harness is secured to the body of the user, press said rigid means against the back of the user, more preferably said rigid member is pressed against is the upper spine region of the back of the user.

Alternatively said rigid member is held apart from the upper spine region of the back of the user, preferably by means of a strut or brace attached to said harness; more preferably the rigid member is not in contact with the upper spine region of the back of the user.

Preferably said rigid member is an elongate member having a first distal end defining the lower end of the substantially vertical member and a second distal end defining the outer end region of said outwardly extending portion thereof.

Preferably said rigid member is an extrusion, preferably plastics, or is of an elongate sheet material shaped, moulded or fabricated to provide said vertically extending region and said outwardly extending region thereof.

Preferably said head supporting region is formed or shaped to additionally provide support to the sides of the head.

Preferably said harness includes or comprises at least two shoulder engaging straps, one each to locate over a shoulder of the user and to locate with said rigid means, or an extension thereof, at the back of said user.

Preferably said shoulder straps are each endless members in use and have a means to open such as a buckle and also to provide an adjustment in the length of said strap.

Preferably said rigid member is provided with lateral extensions, extending from said vertically extending portion of said rigid member from each side thereof each said lateral member having at its distal end thereof a shoulder strap engaging means to allow the location of said shoulder strap with said lateral member.

Preferably each lateral member allows the longitudinal sliding engagement of each shoulder strap at its distal ends thereof, to provide, when said harness is in use a directional stability of said second distal end of said substantially vertical member to prevent it from moving to any significant and functionless degree in a substantially horizonal direction in respect of the head.

Preferably the shoulder strap means each locate to said rigid means at the distal end of said vertically extending member thereof.

Preferably said harness further includes a chest or waist encompassing strap which is located at substantially the point where said shoulder straps locate at said rigid means, and is preferably provided with a release buckle and adjustable means, about the waist or chest of the user.

Preferably the head support is a recreational head support.

Alternatively the head support is for vocational use.

Preferably the head support includes adjustment means capable of altering the tilt of said member, and/or of said second distal end of said substantially vertical member relative to said first distal end.

Preferably the head support includes adjustment means capable of altering the height of the rigid member with respect to the harness, and/or the head support region with respect to the rigid member and/or the harness.

Preferably the head support includes adjustment means capable of altering the distance between said rigid member and said back of said user.

In a further aspect the present invention consists in a head support comprising a rigid member having a vertically extending region and an outwardly extending region supporting the head, when in use in respect of the vertically upright body of a user, in a tilted backwards position to allow viewing of an object above the horizontal, wherein said vertically extending member has a region adapted for engaging with a head support receiving means located at/or near the top of a back pack.

Preferably the head support includes adjustment means capable of altering the tilt of said member, and/or of said second distal end of said substantially vertical member relative to said first distal end.

Preferably the head support includes adjustment means capable of altering the height of the rigid member with respect to the harness, and/or the head support region with respect to the rigid member and/or the harness.

Preferably the head support includes adjustment means capable of altering the distance between said rigid member and said back of said user.

Preferably the head support is a recreational head support.

Alternatively the head support is for vocational use.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples.

### DESCRIPTION OF PREFERRED EMBODIMENT

One preferred form of the present invention will now be described with reference to the accompanying drawings in which:

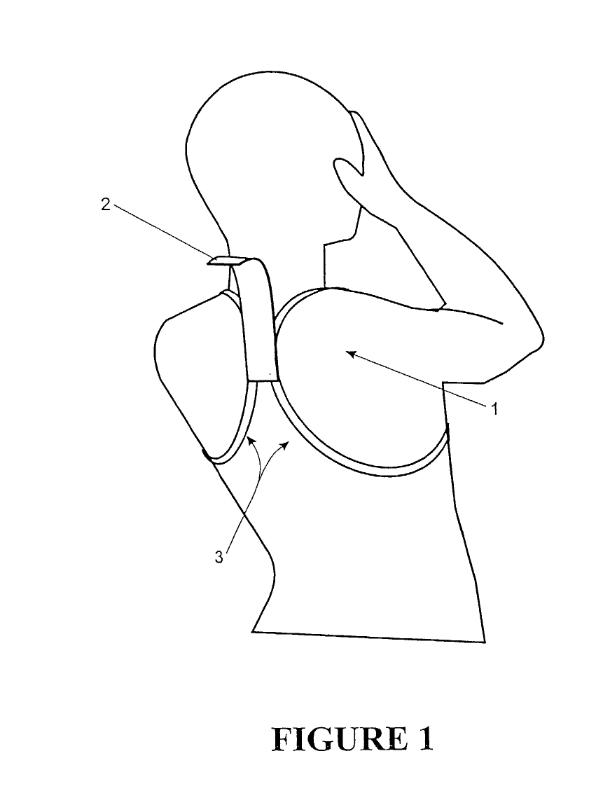

FIG. 1 is a perspective view of the back of a user of the present invention illustrating the head support located with the user in the preferred condition,

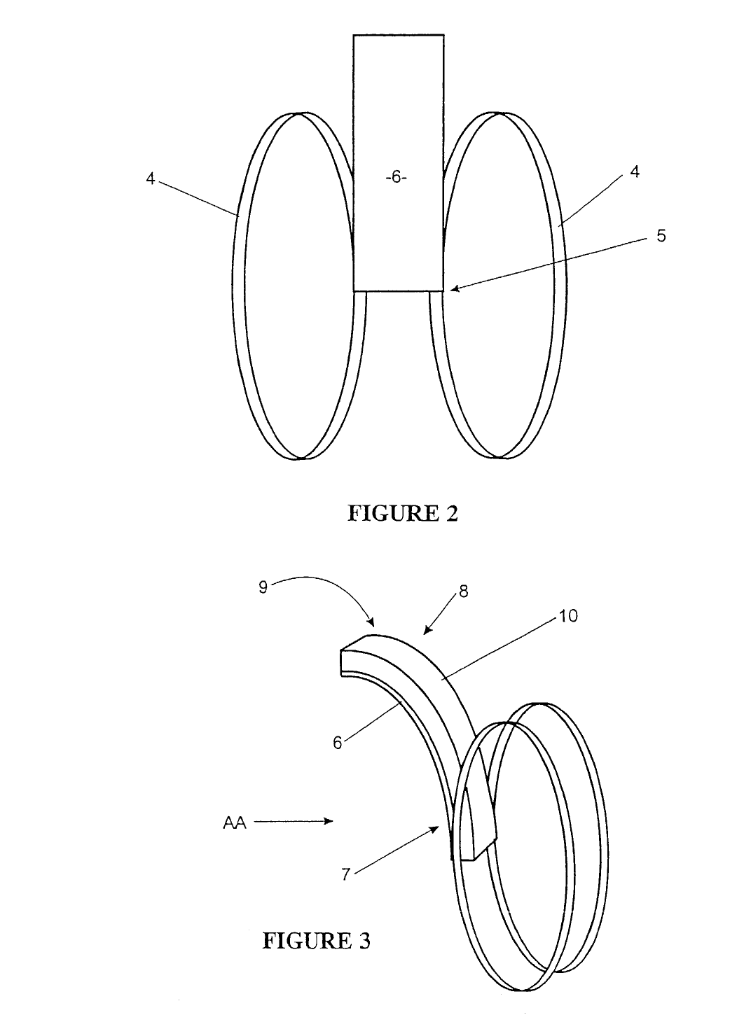

FIG. 2 is a front view in direction AA of FIG. 3 of the head support of the present invention,

FIG. 3 is a perspective view of the head support as shown in FIG. 2,

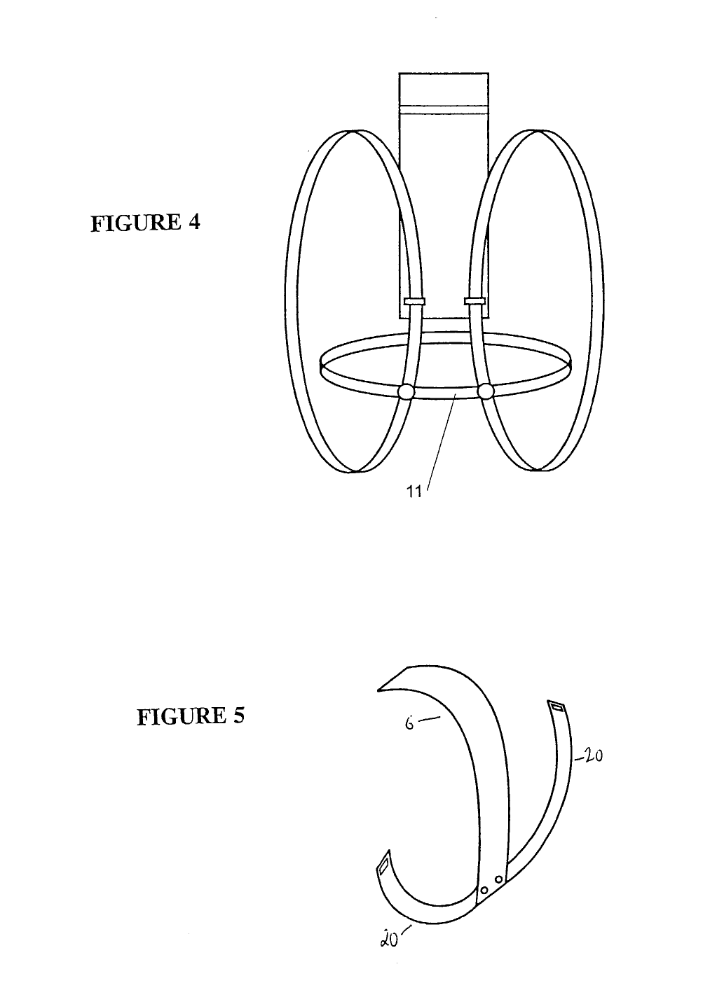

FIG. 4 is a view from the back of an alternative and preferred form of the head support of the present invention

FIG. 5 illustrates the rigid member of the head support in association with a lateral stabilising means with which the straps of the harness can locate.

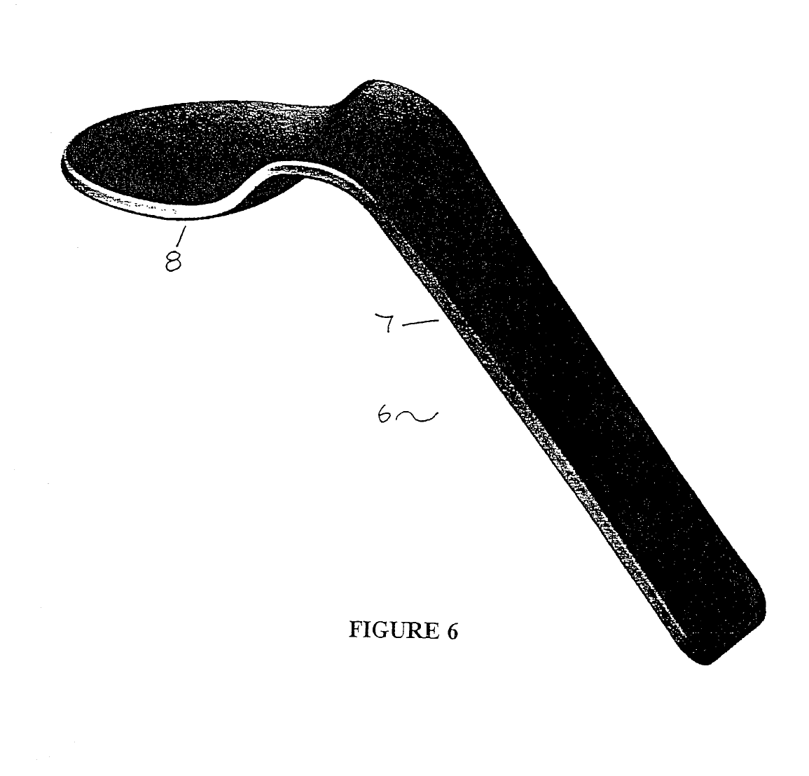

FIG. 6 illustrates a side perspective view of a preferred form of the rigid member of the head support.

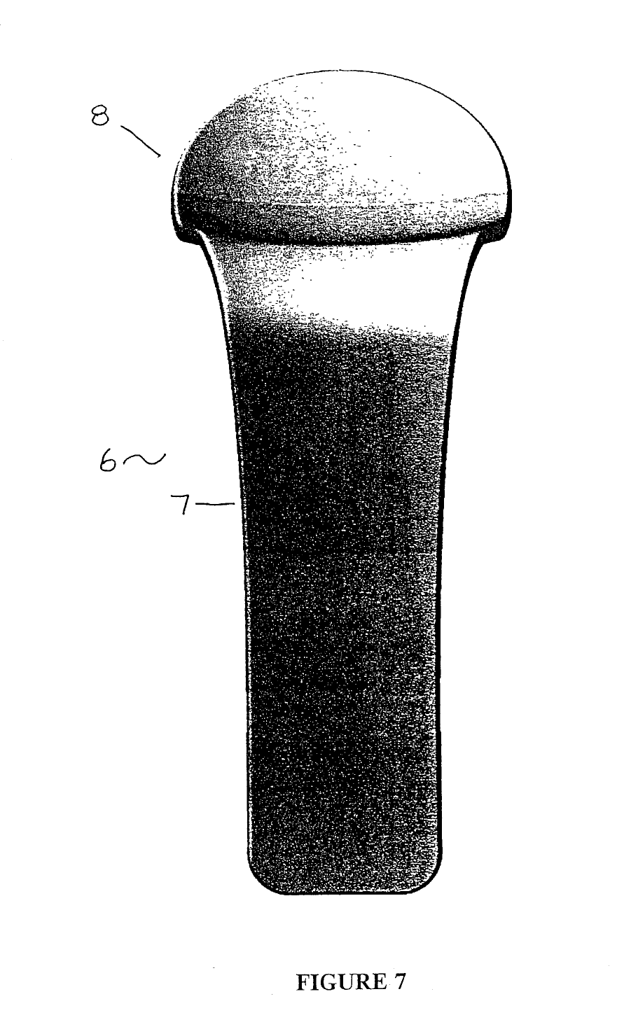

FIG. 7 illustrates a front view in the direction AA of FIG. 3 of the preferred form of FIG. 6.

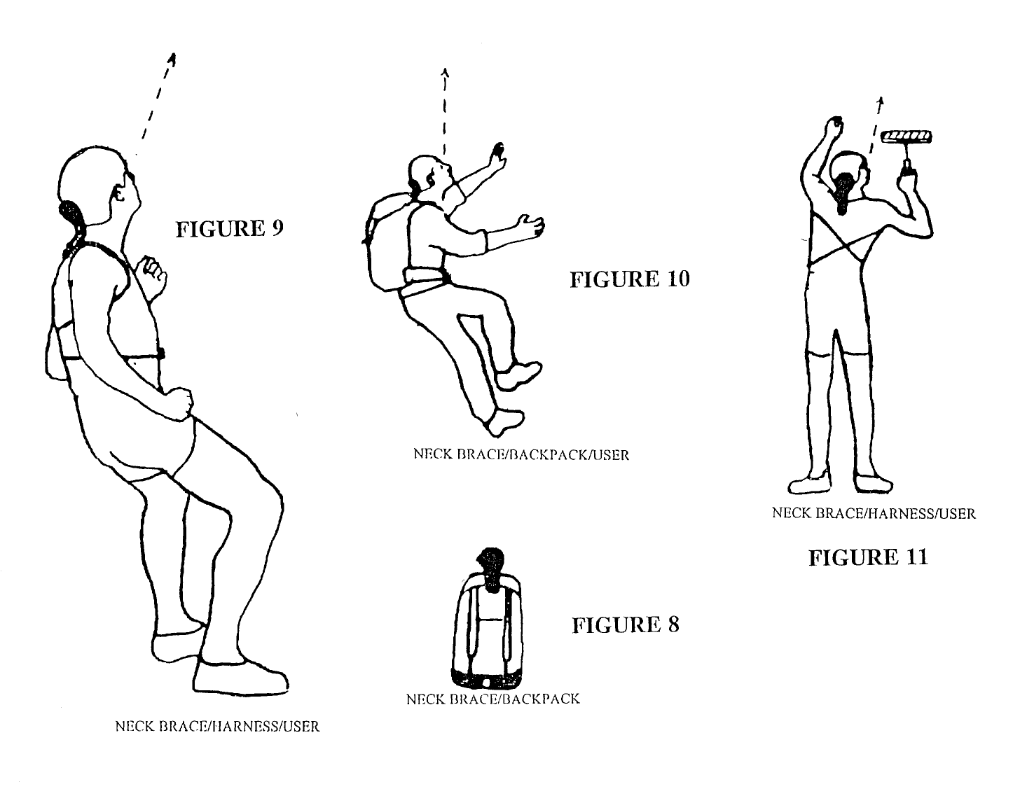

FIG. 8 illustrates a front view in the direction AA of FIG. 3 of an alternative form of the invention, being a back pack.

FIG. 9 illustrates the invention in use by, for example, a belayer FIG. 10 illustrates the invention in use by, for example, a climber.

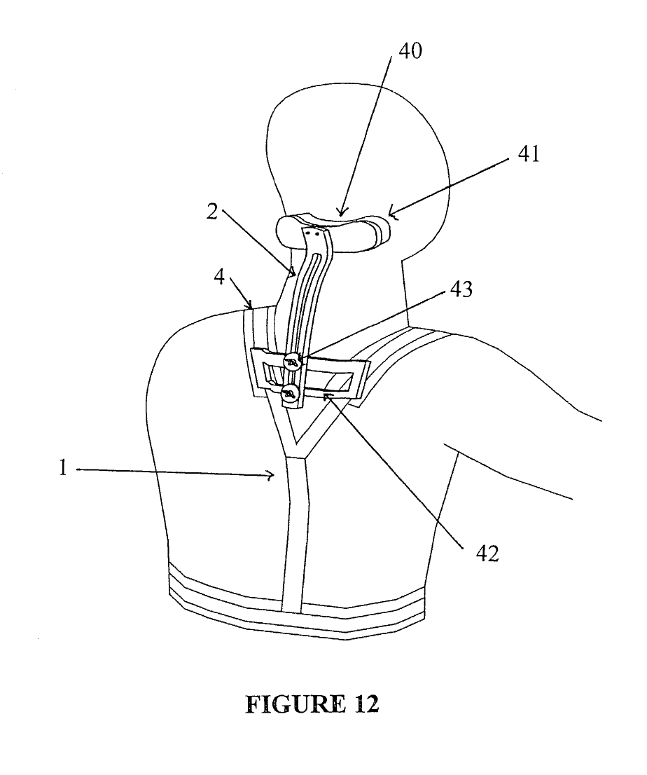

FIG. 11 illustrates the invention in use by, for example, a decorator or painter.

FIG. 12 illustrates the invention according to a further preferred form.

With reference to FIG. 1 there is shown a head support 1 which consists of a rigid member 2 and a harness 3 located or secured with said rigid member.

The rigid member and harness are adapted to be located to the user of the head support of the present invention such that when the user is standing upright and tilts his/her head backwards, there is a region of said rigid member 2 with which the tilted back head can contact and be provided with a vertical support thereby. It is to be noted that the support of the present invention may simply provide neck support for viewing above the horizontal rather than head support, without departing from the scope of the present invention. With reference to FIGS. 2, 9, 10 and 11 there is shown a user of the head support with his head tilted backwards and a part of the head locating against a portion of the rigid member to provide the user with a comfortable and vertical support for the head or neck.

In reference to FIGS. 2 and 3 in the preferred form of the present invention the head support consists of harness which is locatable about the upper body regions of a user of the present invention. The harness is of a configuration which can provide at least vertical stability to the rigid member. It is to be noted that the “harness” can also include a backpack without departing from the scope of the invention. In FIG. 2 in the preferred form there is shown a harness which consists of two shoulder straps 4 which are locatable over the shoulder and underneath the armpits to return back to a region of attachment 5 between the rigid member 6 and the harness 3. In the preferred form the region of attachment is at or towards a lower distal end of the rigid member 6 of the present invention however it is envisaged by the inventor that such location points can vary. With reference to FIG. 1 there is illustrated the shoulder straps 4 running from the region of attachment to the rigid member, over the shoulders under the arms and back towards the region of securement. It will be appreciated by a person skilled in the art that such shoulder straps may be provided with buckles and/or adjustment means to allow for the head support of the present invention to be reconfigured to allow users of different body shapes and sizes to use the particular head support. Furthermore, as discussed the shoulder straps may be those of a backpack configuration.

As indicated in FIGS. 2, 3, 6 and 7 in the preferred form the rigid member 6 is preferably made up from a sheet material such as a moulded plastic, composite or metallic or non-metallic material. The rigid member 6 preferably consists of a vertically extending region 7 and an outwardly extending region 8. The vertically extending region 7 is provided and adapted in association with the harness to lie parallel to and in engagement with the back of a user of the head support of the present invention. Preferably said vertically extending region 7 is of sufficient length to be pressed when said harness is engaged to the user, against the back of the user to provide some stable support in respect of the user of the present invention.

The outwardly extending portion 8 of the rigid member has a head contacting region 9 with which, when said head support is in use, the tilted back head of a user can comfortably located. As indicated in FIGS. 6 and 7, the outwardly extending portion may be concave to allow engagement of the head as shown in FIGS. 3 and 4, for comfort in the wearing of the head support of the present invention there is preferably provided a padded surface 10 on that side of the rigid member which is to be in contact with the body of the user.

FIG. 4 shows and alternative configuration or an additional configuration to the harness of the head support of the present invention. To encourage the rigid means to remain in stable and close contact with the back of a user a further portion of the harness is provided to encompass the chest or waist of the user of the device. This portion 11 may be attached to the shoulder straps 4 and/or be attached to the rigid member of the present invention. Again a person skilled in the art will appreciate that this strap 11 may be provided with a buckle and/or strap length adjusting means to allow a particular head support of the present invention to be adapted for use by different sized and shaped people.

For further support to the rigid means of the present invention a lateral stabiliser may be rigidly attached or form part of the rigid member 6. With reference to FIG. 5 such a lateral stabiliser may for example be arms 20 which extend outwardly (and preferably upwardly) from a region of the rigid member 6. Preferably the region from which the lateral stabilisers extend is a lower or lower most region of the rigid member. The lateral stabilisers are preferably made of a material similar to that of the rigid member or may alternatively be made from any other material and which although preferably deformable, does have its own inherent rigidity. In the preferred form the lateral stabilisers locate at the distal ends with the shoulder straps 4. The shoulder straps will tend to draw the lateral stabiliser to conform with the upper back and shoulder shape of the user of the present invention and hence in the preferred form such lateral stabilisers are of a sheet material or strip wherein the plane of the sheet or strip is in a general parallel direction with the back of the user. Such an orientation of the plane of the lateral stabilisers will allow for it to be conformed or at least in part contoured with the back of the user.

Preferably at the distal ends of the lateral stabiliser there is the provision of the slot through which the shoulder straps 4 can extend while still allowing the distal ends of the lateral stabilisers to have some association with said shoulder straps.

FIG. 12 illustrates an embodiment of a further preferred form of the invention. This Figure includes shows the head support 1 including the rigid member 2, and the shoulder straps 4 of the harness. In this embodiment the head supporting region 40 of the head support 1 is of a substantially arcuate form, providing further support towards the sides of the head of the user. It also includes a padded surface 41 for comfort.

FIG. 12 further illustrates the use of a support or strut 42 which holds and retains the rigid member 2 at a distance from the back of the user. In some situtations (such as on medical or particularly orthopaedic advice) it may be preferable for the rigid member 2 to have no contact with the user's back. This embodiment is appropriate for such situations.

FIG. 12 also illustrates additional adjustment means 43 which may be employed to raise and lower the rigid member and (in this particular embodiment) the head supporting region 40 with respect to the head of the user. It is envisaged than a number of adjustment means may be employed individually or in combination in an embodiment according to the invention, without departing from the scope of the invention. Such adjustment means may be used to raise and and lower the rigid member together with, or separately from the head supporting region; to alter the position of the head supporting region with respect to the rigid member and/or head of the user (in respect of height, and tilt for example) and additionally to alter the distance between the rigid member and/or head supporting region and the back of the head, for example.

1. A head support comprising or including:
a) a harness locatable to the upper body portions of a person
b) a rigid member extending from a region of said harness stabilised in respect of and by the use of the harness relative to the body
wherein, when in use, said harness is attached to the body of a person, said rigid member extending therefrom to provide a support region thereof which when said person is in a substantially upright position, and the head is tilted backwards to allow viewing of an object above the horizontal, said support region comes in contact with at least part of the back of the head and provides at least vertical support to the head.

2. A head support as claimed in claim 1 wherein said rigid member has a vertically extending portion which, in use extends substantially vertically and parallel with the general upright position of said user, and an outwardly extending portion which extends, in use from said vertically extending portion, outwardly from the body of a user wherein at or near the distal end of said outwardly extending portion said head supporting region is provided.

3. A head support as claimed in claim 2 wherein said rigid member is secured to said harness at said vertically extending portion thereof.

4. A head support as claimed in claim 3 wherein said harness is secured to the vertically extending portion of said rigid member to, when said harness is secured to the body of the user, press said rigid member against the back of the user.

5. A head support as claimed in claim 4 wherein said rigid member is pressed against is the upper spine region of the back of the user.

6. A head support as claimed in claim 3 wherein said rigid member is held apart from the upper spine region of the back of the user.

7. A head support as claimed in claim 6 wherein said rigid member is held apart from the upper spine region of the back of the user by means of a strut or brace attached to said harness.

8. A head support as claimed in claim 7 wherein the rigid member is not in contact with the upper spine region of the back of the user.

9. A head support as claimed in claim 5 or claim 8 wherein said rigid member is an elongate member having a first distal end defining the lower end of the substantially vertical member and a second distal end defining the outer end region of said outwardly extending portion thereof.

10. A head support as claimed in claim 9 wherein said rigid member is an extrusion, preferably plastics, or is of an elongate sheet material shaped, moulded or fabricated to provide said vertically extending region and said outwardly extending region thereof.

11. A head support as claimed in claim 10 wherein said head supporting region is formed or shaped to additionally provide support to the sides of the head.

12. A head support as claimed in claim 11 wherein the head support includes one or more adjustment means selected from:
(i) adjustment means capable of altering the tilt of said member, and/or of said second distal end of said substantially vertical member relative to said first distal end;
(ii) adjustment means capable of altering the height of the rigid member with respect to the harness, and/or the head support region with respect to the rigid member and/or the harness;
(iii) adjustment means capable of altering the distance between said rigid member and said back of said user.

13. A head support as claimed claim 12 wherein said rigid member is provided with padding in those regions which, when said head support is in use, are in contact with the body of the user.

14. A head support as claimed in claim 13 wherein said harness includes or comprises at least two shoulder engaging straps, one each to locate over shoulder of the user and to locate with said rigid means, or an extension thereof, at the back of said user.

15. A head support as claimed in claim 14 wherein said shoulder straps are each endless members in use and have a means to open such as a buckle and also to provide an adjustment in the length of said strap.

16. A head support as claimed in claim 15 wherein said rigid member is provided with lateral extensions, extending from said vertically extending portion of said rigid member from each side thereof each said lateral member having at its distal end thereof a shoulder strap engaging means to allow the location of said shoulder strap with said lateral member.

17. A head support as claimed in claim 16 wherein each lateral member allows the longitudinal sliding engagement of each shoulder strap at its distal ends thereof, to provide, when said harness is in use a directional stability of said second distal end of said substantially vertical member to prevent it from moving to any significant and functionless degree in a substantially horizonal direction in respect of the head.

18. A head support as claimed in claim 17 wherein the shoulder strap means each locate to said rigid means at the distal end of said vertically extending member thereof.

19. A head support as claimed in claim 18 wherein said harness further includes a chest or waist encompassing strap which is located at substantially the point where said shoulder straps locate at said rigid means.

20. A head support as claimed in claim 19 wherein said harness further includes a release buckle and adjustable means, about the waist or chest of the user.

21. A head support as claimed in claim 20 wherein the head support is a recreational head support.

22. A head support as claimed in claim 20 wherein the head support is for vocational use.

23. A head support comprising a rigid member having a vertically extending region and an outwardly extending region supporting the head, when in use in respect of the vertically upright body of a user, in a tilted backwards position to allow viewing of an object above the horizontal, wherein said vertically extending member has a region adapted for engaging with a head support receiving means located at/or near the top of a back pack.

24. A head support as claimed in claim 23 wherein the head support includes one or more adjustment means selected from:
(i) adjustment means capable of altering the tilt of said member, and/or of said second distal end of said substantially vertical member relative to said first distal end;
(ii) adjustment means capable of altering the height of the rigid member with respect to the harness, and/or the head support region with respect to the rigid member and/or the harness;
(iii) adjustment means capable of altering the distance between said rigid member and said back of said user.

25. A head support as claimed in claim 24 wherein the head support is a recreational head support.

26. A head support as claimed in claim 24 wherein the head support is for vocational use.
